(19) United States
(12) Patent Application Publication
(10) Pub. No.: US 2025/0359814 A1
(43) Pub. Date: Nov. 27, 2025

(54) SKIN CHARACTERIZATION DEVICE AND METHOD

(71) Applicant: Danvantar Biophotonics Oy, Helsinki (FI)

(72) Inventor: Matti MYLLYMÄKI, Helsinki (FI)

(73) Assignee: Danvantar Biophotonics Oy, Helsinki (FI)

(21) Appl. No.: 18/873,494

(22) PCT Filed: Jun. 13, 2023
(86) PCT No.: PCT/FI2023/050348

§ 371 Date: Dec. 10, 2024

(30) Foreign Application Priority Data
Jun. 22, 2022 (FI) 20225566

(51) Int. Cl. A61B 5/00; A61B 5/01

(52) U.S. Cl. CPC A61B 5/445; A61B 5/0064; A61B 5/0075; A61B 5/01; A61B 5/6844; A61B 2560/0242; A61B 2562/0233; A61B 2562/0271

(57) ABSTRACT

An arrangement which includes a measurement device, and this device includes a radiation-emitting device which illuminates a skin region with radiation in a UV wavelength range, in a visible wavelength range and in a near-infrared wavelength range. The device also includes one or more radiation detectors which measure a reflected radiation spectrum from the skin region in the UV wavelength range, the visible wavelength range and the near-infrared wavelength range.

### FIELD OF THE DISCLOSURE

The present disclosure relates to devices and method for evaluating the condition of a region of skin, and particularly to devices which irradiate the skin surface with non-ionizing radiation.

### BACKGROUND OF THE DISCLOSURE

Visible light and other kinds of non-ionizing radiation can be used to study skin surfaces belonging to humans and animals. It is known that different wavelengths of non-ionizing radiation penetrate to different depths on a skin surface. It is also known that non-ionizing radiation can have beneficial effects on damaged skin. It can, for example, accelerate the healing of an open wound or reduce the harm of psoriasis, eczema or itching skin.

Illuminating the skin with non-ionizing radiation can also produce useful information when the skin surface contains a wound or other damage, for example caused by a skin disease. However, most presently available skin characterization devices illuminate the skin surface with radiation in a narrow wavelength range. The information which can be retrieved from such studies is limited.

Document US20210353148 discloses a device which illuminates skin with ultraviolet, visible and infrared wavelengths. The device also takes images of the skin while it is being illuminated. A high degree of medical expertise is needed for interpreting these images.

### BRIEF DESCRIPTION OF THE DISCLOSURE

An object of the present disclosure is to provide a method and an apparatus which alleviate the above disadvantage. The object of the disclosure is achieved by a method and an arrangement which are characterized by what is stated in the independent claims. The preferred embodiments of the disclosure are disclosed in the dependent claims.

The disclosure is based on the idea of illuminating a region of skin successively with wavelengths ranging from ultraviolet to visible and infrared and measuring the radiation spectrum which is reflected back from this region of skin in each wavelength range.

### BRIEF DESCRIPTION OF THE DRAWINGS

In the following the disclosure will be described in greater detail by means of preferred embodiments with reference to the accompanying drawings, in which

FIG. 1 illustrates a measurement device.

FIGS. 2-4 illustrate methods.

### DETAILED DESCRIPTION OF THE DISCLOSURE

This disclosure describes an arrangement for skin characterization. The arrangement comprises a measurement device, and the measurement device comprises a radiation-emitting device which illuminates a skin region with radiation. The radiation-emitting device also comprises one or more first emitters which emit radiation in a UV wavelength range of 200 nm-400 nm, one or more second emitters which emit radiation in a visible wavelength range of 400 nm-750 nm, and one or more third emitters which emit radiation in a near-infrared wavelength range of 750 nm-900 nm. The device also comprises one or more radiation detectors which measure a reflected radiation spectrum from the skin region as the skin region is illuminated by the radiation-emitting device. The one or more radiation detectors measure the reflected radiation spectrum in the UV wavelength range, the visible wavelength range and the near-infrared wavelength range.

The device may alternatively be a measurement and treatment device for skin characterization and treatment. The arrangement may alternatively be an arrangement for skin characterization and treatment. Any wavelength range presented in this disclosure may extend to the range limit value (for example 400 nm) which has been indicated without including that range limit value, or it may extend to the range limit value and include that value.

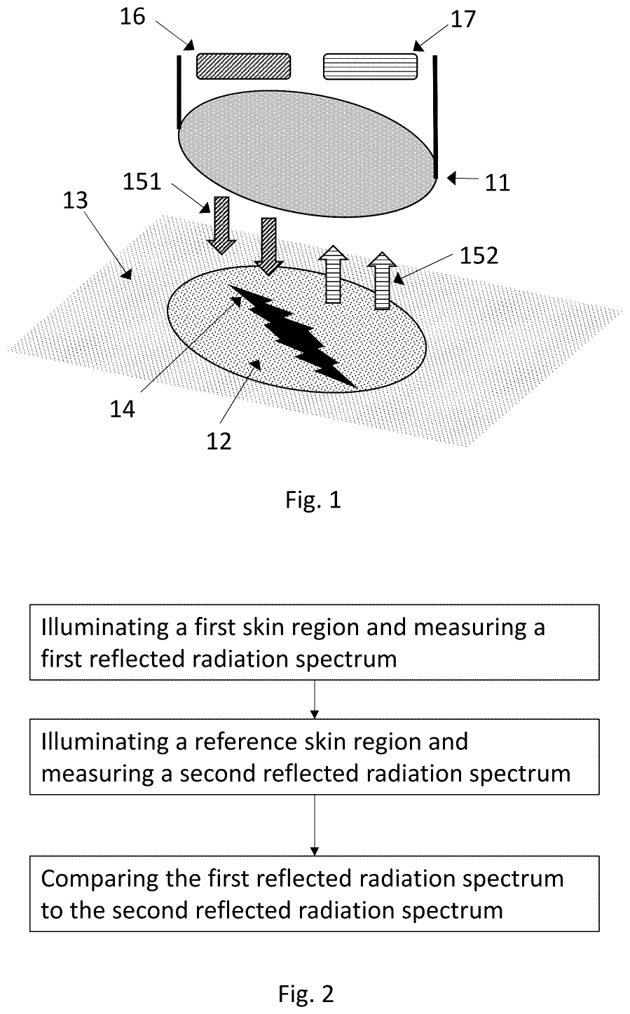

FIG. 1 illustrates schematically a measurement device 11. The measurement device may be a portable device. It may for example weigh less than a kilogram or less than two kilograms so that a user can easily move the device to a desired place on the skin and move it by hand toward and away from the skin surface 13.

The skin region 12 which forms the measurement target for the measurement device may be an area on the skin of a human being. The measurement device 11 illuminates the entire skin region 12 with non-ionizing radiation 151 generated by the radiation-emitting device 16. The radiation detector 17 in the measurement device 11 measures the reflected radiation 152 from the entire skin region 12. A single radiation detector is illustrated, but many radiation detectors can also be used. The radiation-emitting device 16 and the radiation detector 17 have for simplicity been illustrated on opposite sides of the measurement device 11 in FIG. 1. However, any geometry can be used. The radiation-emitting device could for example surround the radiation detector, or vice versa.

Additional optional parts of the arrangement and the measurement device are presented below. Any options may be combined with each other.

The reflected radiation spectrum may comprise data which relates the intensity (expressed for example in W/cm²) of the reflected radiation to radiation wavelength. For example, if the one or more first emitters successively illuminate the skin region 12 with radiation 151 which has a first UV wavelength, then a second UV wavelength and then a third UV wavelength, the radiation detector 17 may measure the intensity of the reflected radiation 152 at each of these UV wavelengths. The illumination may be performed in a similar manner in the visible wavelength range and the near-infrared range, and the radiation detector 17 may measure the radiation intensity at each wavelength in the succession to produce the reflected radiation spectrum data.

The one or more first emitters (which are not separately illustrated in FIG. 1) may for example comprise a light-emitting diode or a laser. The one or more second emitters may for example comprise a light-emitting diode or a laser. The one or more third emitters may for example comprise a light-emitting diode or a laser.

The one or more first radiation emitters may for example comprise at least one first radiation emitter which exhibits an emission peak in the UVA (320-400 nm) range, and/or at least one first radiation emitter which exhibits an emission peak in the UVB (280-320 nm) range, and/or at least one first radiation emitter which exhibits an emission peak in the UVC (200-280 nm) range. The one or more second radiation emitters may comprise at least one second radiation emitter which exhibits an emission peak in the blue (440-510 nm) wavelength range, and/or at least one first radiation emitter which exhibits an emission peak in the red (610-760 nm) wavelength range. The first, second and third radiation emitters may all be selected so that they together cover sufficiently well the entire wavelength range of interest.

The one or more radiation detectors may for example comprise a single spectrometer which measures the reflected radiation spectrum across a broad range of wavelengths. Alternative, they may comprise multiple radiation detectors, and each of these radiation detectors may measure the part of the reflected radiation spectrum in a specific wavelength range. The radiation detector may be any kind of photodetector or, in the case of the near-infrared range, any kind of bolometer. The one or more radiation detectors may alternatively comprise a UV photodetector for measuring reflected radiation spectrum in the UV wavelength range, a spectrometer or photodetector for measuring the reflected radiation spectrum in the visible wavelength range, and a photodetector or bolometer for measuring the reflected radiation spectrum in the near-infrared range.

The measurement device may comprise an opening, and the skin region may be illuminated through this opening so that the size and position of the opening, and the place where the measurement device is held, determine the size and position skin region which is studied. The opening may be covered with a transparent window.

Alternatively, the skin region which is illuminated may be determined by a protective shield which is placed on the surface of the skin. An opening in this shield may determine the skin region which is being studied.

The area of the skin region may for example be in the range 0.1 mm²-100 cm². Alternatively, the areal size of the skin region may be in any of the ranges 0.1 mm²-1 mm², 0.1 mm²-10 mm², 0.1 mm²-1 cm², 0.1 mm²-10 cm², 0.01 cm²-1 cm², 0.05 cm²-5 cm², 0.05 cm²-10 cm², 1 cm²-50 cm², 5 cm²-100 cm², 10 cm²-100 cm², 20 cm²-100 cm², 50 cm²-100 cm² or 5 cm²-50 cm².

Skin damage 14 may lie at least partly within the skin region 12. The skin damage may be an open wound, or it may be other kind of damage caused by psoriasis or other skin diseases. However, the skin region 12 may alternatively contain fully healthy skin. Measurements performed on healthy skin may be used as a comparative reference to measurements which are performed on damaged skin.

The measurement device may comprise a proximity sensor (not illustrated) for measuring the distance between the skin region 12 and the radiation-emitting device 11. The proximity sensor may be configured to monitor this distance while the skin region is illuminated and the reflected radiation spectrum is measured. Users who hold the measurement device in their hand while the measurement is being performed may not be able to hold their hand steady throughout the measurement. They may also misjudge the distance which a user manual recommends to be used for a given measurement. The data provided by the proximity sensor can be used to automatically adjust the intensity of the emitted radiation so that it is appropriate for the intended measurement.

In other words, the control unit which is described in more detail below may be configured to automatically adjust the distance between the illuminated skin region and the radiation emitters. Optionally, the radiation-emitting device and the control unit may be configured so that the distance of the first, second and third emitters to the skin region may be adjusted independently of each other.

The device may comprise a temperature sensor for measuring the ambient temperature. This measurement may be conducted when the skin region is illuminated. The device May further comprise a humidity sensor for measuring the ambient humidity. This measurement may also be conducted while the skin region is illuminated. The ambient temperature and humidity are parameters which may influence the measurement results and the treatments that may be performed based on the measurement. The device may further comprise a 3D camera for measuring the topography of the skin region and/or the geometry and topography of any particular object which lies in the skin region, such as a wound. The device may also comprise a colour sensor for measuring the colour of the skin region. The proximity sensor, temperature sensor, humidity sensor, 3D camera and colour sensor may be implemented independently of each other. In other words, only one of them may be used, or any two of them may be used, or any combination of three of them, or four of them.

The arrangement described in this disclosure may comprise a computer with a control unit which controls the measurement and performs the desired computational analysis. The control unit may be located in the measurement device. Alternatively, the control unit may be physically separate from the measurement device and located for example on a mobile phone, tablet computer, personal computer or the like, which may control and communicate with the measurement device without user intervention, for example via a wireless data link such as Bluetooth, Wifi, GSM/3G/4G or via a wired data link. In the latter case, the arrangement comprises both the control unit and the measurement device, even though they are physically separated from each other.

The control unit may comprise one or more data processors. The control unit may be connected to a memory unit where computer-readable data or programs can be stored. The memory unit may comprise one or more units of volatile or non-volatile memory, for example EEPROM, ROM, PROM, RAM, DRAM, SRAM, firmware, programmable logic, etc.

The methods described in the present disclosure may be implemented in, for example, hardware, software, firmware, special purpose circuits or logic, a computing device or some combination thereof. Software routines, which may also be called program products, are articles of manufacture and can be stored in any apparatus-readable data storage medium, and they include program instructions to perform particular predefined tasks. Accordingly, embodiments of this invention also provide a computer program product, readable by a computer and encoding instructions for performing the methods described in this disclosure.

The control unit may be configured to communicate commands to the measurement device to perform a measurement method and to retrieve measured data from the measurement device. Some possible methods are described below. The memory unit may contain data which the control unit uses for analyzing data measured by the measurement device and making selections based on such analyses. The control unit may be connected to an interface unit which allows an operator to operate the measurement device. The selections recommended by the control unit may be transmitted to the operator through the interface unit, and the operator may be able to start a given measurement or treatment by sending commands to the control unit via the interface unit.

The control unit may for example be configured to (1) command the measurement device to illuminate the skin region with radiation in the UV wavelength range, the visible wavelength range and the near-infrared wavelength range and to measure a reflected radiation spectrum from the skin region when the skin region is illuminated in these three wavelength ranges, (2) select one or more treatment wavelengths and corresponding one or more treatment intensities and one or more treatment durations based on the reflected radiation spectrum, and (3) command the measurement device to illuminate the skin region with radiation at each of the one or more treatment wavelengths at the corresponding one or more treatment intensities for a duration which equals the corresponding treatment duration. Additional commands may be added as needed.

Appropriate emission wavelengths and illumination durations may be tabulated and stored in a memory unit. However, adjustments to the predetermined characterization or treatment plan may have to be made dynamically. The person being treated may for example move her body, so that the position of the measurement device is shifted. The arrangement described in this disclosure may optionally comprise a safety functionality. The purpose of this functionality may be to continuously assess the radiation dose received by the skin region and to adjust the radiation exposure if necessary. The safety functionality may be based on measurements performed by the measurement device.

The safety functionality may be used when radiation in the UV wavelength range is emitted. It may also be used when radiation in the visible or near-infrared wavelength range is emitted. The control unit may be configured to implement the safety function by retrieving measurement data from the one or more radiation detectors, and/or from a temperature sensor, and/or from a colour sensor, and/or from a proximity sensor, when a skin region is being illuminated.

When performing the safety function, the control unit may be configured to compare the data retrieved from any or all of the above-mentioned detectors and sensors to reference values stored in a safety protocol in a memory unit. The control unit may be configured to perform a safety action if the retrieved data differs from the reference values by more than a predetermined threshold. The safety action may for example comprise reducing the radiation intensity with which the skin region is being illuminated, or shutting off one or more of the first, second or third emitters either temporarily or for the remaining duration of the characterization/treatment. Alternatively, the safety action may comprise automatically moving the measurement device further away from the skin region.

The reference values may be based on earlier experiments and recommendations which aim to ensure that the radiation dose absorbed by the skin region does not exceed a safety limit.

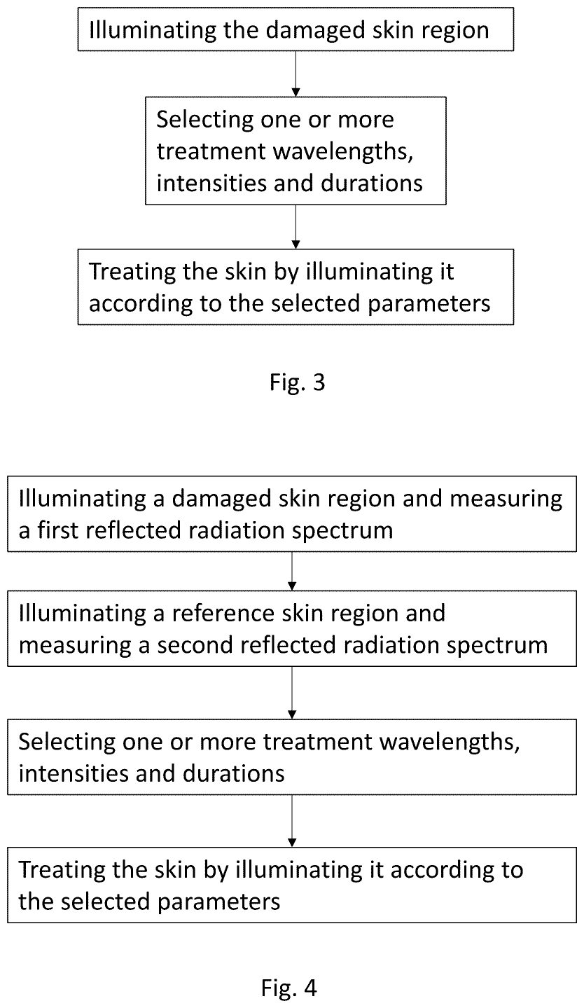

A method for characterizing skin may comprise a first step where a first skin region is illuminated in a UV wavelength range of 200 nm-400 nm, and with radiation in a visible wavelength range of 400 nm-750 nm and with radiation in a near-infrared wavelength range of 750 nm-900 nm. A first reflected radiation spectrum may be measured in the same first step from the first skin region when the first skin region is illuminated in these three wavelength ranges. In a second step, which may be performed before or after the first step, a reference skin region may be illuminated with radiation in the UV wavelength range, the visible wavelength range and the near-infrared wavelength range, and a second reflected radiation spectrum may be measured from the reference skin region when the reference skin region is illuminated in these three wavelength ranges. In a third method step, the first reflected radiation spectrum may be compared to the second reflected radiation spectrum. This method has been illustrated in FIG. 2. The method can be performed with the device which was presented above, and all device options presented above can be used when the method is implemented. The third method step may for example comprise subtraction of the first reflected radiation spectrum from the second reflected radiation spectrum. The method for characterizing skin may alternatively be a method for assisting skin diagnosis or a method for monitoring the result of skin treatment.

The method for characterizing skin described above may be expanded to a method for selecting skin treatment by including a fourth method step where one or more treatment wavelengths and corresponding one or more treatment intensities and treatment durations are selected based on the comparison performed in the third step.

The first and second reflected radiation spectra may be measured from the entire first skin region and the entire reference skin region. The first skin region and the reference skin region may have any of the areal sizes that were mentioned above. The size of the first skin region may be substantially equal to the size of the reference skin region. The device described above may be utilized to perform this method. The wavelength options presented above for the one or more first emitters, one or more second emitters and one or more third emitters can all be applied in this method as well. The options described in this paragraph apply also to the method described in the next paragraph.

A method for treating a damaged skin region with a radiation-emitting device may comprise the steps of (1) illuminating the damaged skin region with radiation in a UV wavelength range of 200 nm-400 nm, with radiation in a visible wavelength range of 400 nm-750 nm and with radiation in a near-infrared wavelength range of 750 nm-900 nm and measuring a first reflected radiation spectrum from the damaged skin region when the damaged skin region is illuminated in these three wavelength ranges. (2) Selecting one or more treatment wavelengths and corresponding one or more treatment intensities and one or more treatment durations based on the first reflected radiation spectrum. (3) Treating the skin by illuminating the skin region with radiation at each of the one or more treatment wavelengths at the corresponding one or more treatment intensities for a duration which equals the corresponding treatment time. This method is illustrated in FIG. 3. The method can be performed with the device which was presented above, and all device options presented above can be used when the method is implemented. In all methods described in this disclosure, the device may be placed at a suitable distance from the skin surface when the method is performed. This distance may be monitored and radiation intensity may be adjusted if the distance changes.

Optionally, the selection of the one or more treatment wavelengths and corresponding one or more treatment intensities may be based also on a second reflected radiation spectrum measured from a reference skin region, as described below.

The one or more treatment wavelengths may for example be selected as follows. If the first reflected radiation spectrum shows a trough (negative peak) at wavelength L1, then the damaged skin region has absorbed a significant amount of radiation at this wavelength. It may then be desirable to illuminate the damaged skin region with a radiation wavelength which lies as close as possible to L1. The selection of the one or more treatment wavelengths may then for example include a decision to illuminate the damaged skin region with an emitter which has an emission peak at wavelength L2 which lies closer to L1 than the emission peak wavelengths of the other emitters in the radiation-emitting device. L2 is in this case a selected treatment wavelength. Multiple treatment wavelengths may be selected with the same principle if the first reflected radiation spectrum comprises multiple troughs.

The control unit may be configured to perform the selection of the one or more treatment wavelengths and corresponding one or more treatment intensities automatically, without simultaneous human intervention in the selection process.

Other measured parameters may also influence the selection of treatment wavelengths, intensities and durations. If, for example, the measurement data from the 3D camera indicates that there is a deep wound in the skin region, then some possible treatment wavelengths must be excluded because they could have a damaging effect on the wound. The decision described above can therefore involve many different considerations and steps where all the available data is used to maximize the beneficial effects while avoiding harm.

The treatment effect of radiation depends on its wavelength range. In the UV wavelength the radiation may disinfect wounds and deactivate infections. In the visible wavelength range the radiation may deactivate T-cell and dendrite cells. In the visible wavelength range and in the near-infrared wavelength range the radiation may energize mitochondria and increase the production of collagen. Many other effects are also possible.

A treatment intensity (expressed for example in mW/cm²) and a treatment duration (expressed for example in seconds) may be selected for each selected treatment wavelength. The selection of both the intensity and the duration may for example be based on the measurement data (the first radiation spectrum) retrieved by the control unit from the measurement device. It may also be based on treatment recommendation data stored in the memory unit. The control unit may be configured to compare the measurement data to the treatment recommendation data to determine each treatment intensity and treatment duration. The treatment recommendation data may have been compiled before the measurement device was taken into use. Alternatively or complementarily, the treatment recommendation data may have been at least partly compiled or modified by measurement data which was retrieved by the control unit in previous measurements performed with the measurement device.

The treatment intensity may for example be in the range of 1 mW/cm²-20 000 mW/cm², or in any of the ranges 1 mW/cm²-10 000 mW/cm², 1 mW/cm²-1000 mW/cm², 1 mW/cm²-100 mW/cm², 10 mW/cm²-20 000 mW/cm², 10 mW/cm²-10 000 mW/cm², 10 mW/cm²-1000 mW/cm², 10 mW/cm²-100 mW/cm², 100 mW/cm²-20 000 mW/cm², 100 mW/cm²-10 000 mW/cm², or 100 mW/cm²-1000 mW/cm². The treatment durations may for example be in the range of 10 seconds-30 minutes.

The method for treating a damaged skin region may optionally also comprise the steps of (1b) illuminating a reference skin region with radiation in the UV wavelength range, the visible wavelength range and the near-infrared wavelength range, and measuring a second reflected radiation spectrum from the reference skin region when the reference skin region is illuminated in these three wavelength ranges, and (2b) selecting the one or more treatment wavelengths and corresponding one or more treatment intensities and one or more treatment durations also based on the second reflected radiation spectrum. The selection may be, but does not necessarily have to be, based on a comparison between the first and second reflected radiation spectra. This method is illustrated in FIG. 4.

The one or more treatment wavelengths may then for example be selected as follows. The first reflected radiation spectrum may be subtracted from the second reflected radiation spectrum to produce a third spectrum. If the third spectrum shows a peak or a trough (negative peak) at wavelength L3, then the selection of the one or more treatment wavelengths may for example include a decision to illuminate the damaged skin region with an emitter which has an emission peak at wavelength L4 which lies closer to L3 than the emission peak wavelengths of the other emitters in the radiation-emitting device. L4 is in this case a selected treatment wavelength. Multiple treatment wavelengths may be selected with the same principle if the third radiation spectrum comprises multiple peaks and/or troughs. The treatment intensity and duration may be selected with the procedures described above.

Both the method for characterizing skin and the method for treated a damaged skin region may also comprise the step of measuring a distance between the skin region and the radiation-emitting device when the skin is being illuminated, and the step of adjusting the illumination intensity if the measured distance changes. In other words, the control unit may be configured to continuously receive data from a proximity sensor when a measurement or treatment step if being performed. The control unit may be configured to adjust the radiation intensity or treatment intensity emitted by the radiation-emitting device if the distance between the radiation-emitting device and the skin region changes before a measurement or treatment step has been finished. Through this adjustment, the radiation intensity which strikes the skin surface can be kept constant even if the distance changes due to human error.

Both the method for characterizing skin and the method for treating a damaged skin region may also comprises the step of measuring the ambient temperature and/or the step of measuring the ambient humidity. Each method may also comprise the step of measuring the topography of the skin region, and/or the step of measuring the colour of the skin region. Finally, the temperature of the skin region may also be determined or measured from the part of the reflected radiation spectrum which lies in the near-infrared wavelength.

All measurement parameters mentioned above may also be monitored during the treatment. In other words, the method for treating a damaged skin region may comprise the additional step of measuring one or more of these parameters when the treatment is in progress.

1. An arrangement for skin characterization, wherein the arrangement comprises a measurement device, and the measurement device comprises a radiation-emitting device which illuminates a skin region with radiation, and the radiation-emitting device comprises
one or more first emitters which emit radiation in a UV wavelength range of 200 nm-400 nm,
one or more second emitters which emit radiation in a visible wavelength range of 400 nm-750 nm,
one or more third emitters which emit radiation in a near-infrared wavelength range of 750 nm-900 nm,
wherein the device also comprises one or more radiation detectors which measure a reflected radiation spectrum from the skin region as the skin region is illuminated by the radiation-emitting device, and the one or more radiation detectors measure the reflected radiation spectrum in the UV wavelength range, the visible wavelength range and the near-infrared wavelength range.

2. The arrangement according to claim 1, wherein the device comprises a proximity sensor for measuring the distance between the skin region and the radiation-emitting device.

3. The arrangement according to claim 1, wherein the device comprises a temperature sensor for measuring the ambient temperature.

4. The arrangement according to claim 1, wherein the device comprises a humidity sensor for measuring the ambient humidity.

5. The arrangement according to claim 1, wherein the device comprises a 3D camera for measuring the topography of the skin region.

6. The arrangement according to claim 1, wherein the device comprises a colour sensor for measuring the colour of the skin region.

7. A method for characterizing skin, characterized in that the method comprises the steps of:
illuminating a first skin region in a UV wavelength range of 200 nm-400 nm, and with radiation in a visible wavelength range of 400 nm-750 nm and with radiation in a near-infrared wavelength range of 750 nm-900 nm, and measuring a first reflected radiation spectrum from the first skin region when the first skin region is illuminated,
illuminating a reference skin region with radiation in the UV wavelength range, the visible wavelength range and the near-infrared wavelength range, and measuring a second reflected radiation spectrum from the reference skin region when the reference skin region is illuminated, and
comparing the first reflected radiation spectrum to the second reflected radiation spectrum.

8. The method according to claim 7, wherein the method comprises the step of measuring a distance between the skin region and the radiation-emitting device when the skin is being illuminated, and the step of adjusting the illumination intensity if the measured distance changes.

9. The method according to claim 7, wherein the method comprises the step of measuring the ambient temperature.

10. The method according to claim 7, wherein the method comprises the step of measuring the ambient humidity.

11. The method according to claim 7, wherein the method comprises the step of measuring the topography of the skin region.

12. The method according to claim 7, wherein the method comprises the step of measuring the colour of the skin region.

13. The method according to claim 7, wherein the method comprises the step of measuring the temperature of the skin region

14. A computer program product readable by a computer, wherein the computer program product comprising a non-transitory computer readable medium with machine readable instructions to perform the method according to claim 7.
